(19) United States
(12) Patent Application Publication
(10) Pub. No.: US 2002/0001843 A1
(43) Pub. Date: Jan. 3, 2002

(54) Starch biosynthetic enzymes

(76) Inventors: Jonathan E. Lightner, Mulino, OR (US); Stephen M. Allen, Wilmington, DE (US); Catherine J. Thorpe, St. Albans (GB)

Correspondence Address: E I DU PONT DE NEMOURS AND COMPANY, LEGAL DEPARTMENT - PATENTS, 1007 MARKET STREET, WILMINGTON, DE, 19898, US

(21) Appl. No.: 09/829,482

(22) Filed: Apr. 10, 2001

Related U.S. Application Data
(62) Division of application No. 09/073,297, filed on May 6, 1998, now Pat. No. 6,255,114.

(51) Int. Cl. C12N 15/63; A61K 31/70; C12N 15/85; C12N 15/87; A01N 43/04

(52) U.S. Cl. 435/455; 514/44

(57) ABSTRACT

This invention relates to isolated nucleic acid fragments encoding all or a substantial portion of a plant glycogenin or water stress protein. The invention also relates to the construction of chimeric genes encoding all or a portion of a plant glycogenin or water stress protein, in sense or antisense orientation, wherein expression of the chimeric gene results in production of altered levels of a plant glycogenin or water stress protein in a transformed host cell.

### FIELD OF THE INVENTION

This invention is in the field of plant molecular biology. More specifically, this invention pertains to nucleic acid fragments encoding enzymes involved in starch biosynthesis in plants and seeds.

### BACKGROUND OF THE INVENTION

Starch is an important component of food, feed, and industrial products. Broadly speaking, it consists of two types of glucan polymers: relatively long chained polymers with few branches known as amylose, and shorter chained but highly branched molecules called amylopectin. Its biosynthesis depends on the complex interaction of multiple enzymes (Smith, A. et al., (1995) Plant Physiol. 107:673-677; Preiss, J., (1988) Biochemistry of Plants 14:181-253). Chief among these are ADP-glucose pyrophosphorylase, which catalyzes the formation of ADP-glucose; a series of starch synthases which use ADP glucose as a substrate for polymer formation using α-1-4 linkages; and several starch branching enzymes, which modify the polymer by transferring segments of polymer to other parts of the polymer using α-1-6 linkages, creating branched structures. However, based on data from starch forming plants such as potato, and corn, it is becoming clear that other enzymes also play a role in the determination of the final structure of starch. In particular, debranching and disproportionating enzymes not only participate in starch degradation, but also in modification of starch structure during its biosynthesis. Different models for this action have been proposed, but all share the concept that such activities, or lack thereof, change the structure of the starch produced.

This is of applied interest because changes in starch structure, such as the relative amounts of amylose and amylopectin or the degree and length of branching of amylopectin, alter its function in cooking and industrial processes. For example, starch derived from different naturally occurring mutants of corn can be shown on the one hand to differ in structure and correspondingly to differ in functional assays such as Rapid Visco analysis, which measures changes in viscosity as starch is heated and then cooled (Walker, C. E., (1988) Cereal Foods World 33:491-494). The interplay of different enzymes to produce different structures, and in turn how different structures correlate with different functionalities, is not yet completely understood. However, it is understood that changing starch structure will result in alteration in starch function which can in turn lead to new applications or reduced processing costs (certain starch functionalities can at present only be attained through expensive chemical modification of the starch).

Glycogen, a non-plant analogue of starch, is synthesized by the concerted actions of glycogen synthase and glycogen branching enzymes in much the same way that starch biosynthesis occurs in plants. Glycogen synthesis requires a primer for the initial action of the glycogen synthase enzyme. This primer function is thought to be provided by a self-glucosylating protein called glycogenin in mammals. Inactivation of the two genes that encode this enzyme in yeast has been shown to result in the absence of glycogen. It is evident that a similar primer function may be necessary for starch biosynthesis in plants and the isolation of such a self glucosylating activity has been the subject some study (Singh, D. G. et al., (1995) FEBS Letters 376:61-64; World Patent Publication No. WO 94/04693). These reports describe the identification and purification a self-glucosylating protein activity from plants that is structurally unrelated to glycogenin. However, these reports provide no direct evidence that this protein is essential for starch biosynthesis. Lastly, the rice gene WSI76 is a gene induced by short term water stress. Its expression is decreased in response to chilling (Plant Mol Biol 1994 Oct;26(l):339-352). WS176 may be a rice glycogenin because its only homology to a functionally characterized protein is to glycogenin.

Alterations in starch fine structure are known to result in changes to the physiochemical properties of the starch. Because starch fine structure results from the concerted action of several starch synthases, starch branching enzymes and starch debranching enzymes, it is reasonable to suppose that manipulating the amount of substrate for these enzymes may impact on the ultimate structure of the starch granule. Further it is clear that attempts to manipulate starch fine structure through altering expression of starch biosynthetic genes may lower the overall production of starch by reducing the amount of substrate, glucan chains, available to prime synthesis. One useful approach to resolve such difficulties would be the overexpression of a primer protein, glycogenin. Finally, manipulating the expression of the glycogenin primer may be used, for example, to alter the total number of granules initiated in corn endosperm. Increasing or decreasing the number of initial primers for synthesis might reasonably be expected to decrease or increase, respectively, the ultimate size of the synthesized granules. Altering granule size may usefully alter starch functionality and or starch.

The role of glycogenin in starch biosynthesis suggests that over-expression or reduction of expression of genes encoding glycogenin in corn, rice or wheat could be used to alter branch chain distribution of the starch produced by these plants. While glycogenin genes and genes encoding peptides with homology to glycogenin have been described from other organisms (Barbetti, F. et al. (1995) Diabetologia 38:295; Wilson, R. et al. (1994) Nature 368:32-38; Takahashi, R. et al. (1994) Plant Mol. Biol. 26(l):339-352), a glycogenin gene has yet to be described for corn, rice or wheat.

### SUMMARY OF THE INVENTION

The instant invention relates to isolated nucleic acid fragments encoding corn, rice and wheat glycogenin and water stress proteins. In addition, this invention relates to nucleic acid fragments that are complementary to nucleic acid fragments encoding corn, rice and wheat glycogenin and water stress proteins.

In another embodiment, the instant invention relates chimeric genes encoding a corn, rice and wheat glycogenin and water stress protein or nucleic acid fragments that are complementary to nucleic acid fragments encoding a corn, rice and wheat glycogenin and water stress protein, operably linked to suitable regulatory sequences, wherein expression of the chimeric gene results in production of altered levels of a corn, rice and wheat glycogenin or water stress protein in a transformed host cell.

In a further embodiment, the instant invention concerns a transformed host cell comprising in its genome a chimeric gene encoding corn, rice and wheat glycogenin or water stress protein, operably linked to suitable regulatory sequences, wherein expression of the chimeric gene results in production of altered levels of corn, rice and wheat glycogenin or water stress protein in the transformed host cell. The transformed host cells can be of eukaryotic or prokaryotic origin, and include cells derived from higher plants and microorganisms. The invention also includes transformed plants that arise from transformed host cells of higher plants, and from seeds derived from such transformed plants.

An additional embodiment of the instant invention concerns a method of altering the level of expression of a corn, rice and wheat glycogenin or water stress protein in a transformed host cell comprising: a) transforming a host cell with the chimeric gene encoding a corn, rice and wheat glycogenin or water stress protein, operably linked to suitable regulatory sequences; and b) growing the transformed host cell under conditions that are suitable for expression of the chimeric gene wherein expression of the chimeric gene results in production of altered levels of a corn, rice and wheat glycogenin and water stress protein in the transformed host cell.

An addition embodiment of the instant invention concerns a method for obtaining a nucleic acid fragment encoding all or substantially all of an amino acid sequence encoding a plant glycogenin.

### BRIEF DESCRIPTION OF THE DRAWINGS AND SEQUENCE DESCRIPTIONS

The invention can be more fully understood from the following detailed description and the accompanying drawings and the sequence descriptions which form a part of this application.

FIG. 1 shows a comparison of the amino acid sequences of human glycogenin (U44131), a Caenorhabditis elegans glycogenin homolog (Z82052) and the instant corn glycogenin enzyme (cc3.mn0001.f7).

FIG. 2 shows a comparison of the amino acid sequences of the instant corn glycogenin enzyme (cc3.mn0001.f7) and two related plant sequences: a conceptual translation of a portion of a genomic clone from Arabidopsis thaliana (1922956) with homology to glycogenin, and a rice (Oryza sativa) protein induced by water stress (D26537).

FIG. 3 is a digitized image of a stained SDS-PAGE gel demonstrating expression of the instant corn glycogenin in E. coli. “Soluble” indicates that the analyzed samples were obtained from the soluble fraction of the cell extract. “Pellet” indicates that the analyzed samples were obtained from the insoluble fraction of the cell extract. A “+” sign indicates that the analyzed samples were extracted from E. coli transformants harboring an expression vector comprising the PCR generated EST cc3.mn0001.f7 insert. “Control” indicates that the analyzed samples were extracted from E. coli transformants harboring an empty pET24d expression vector.

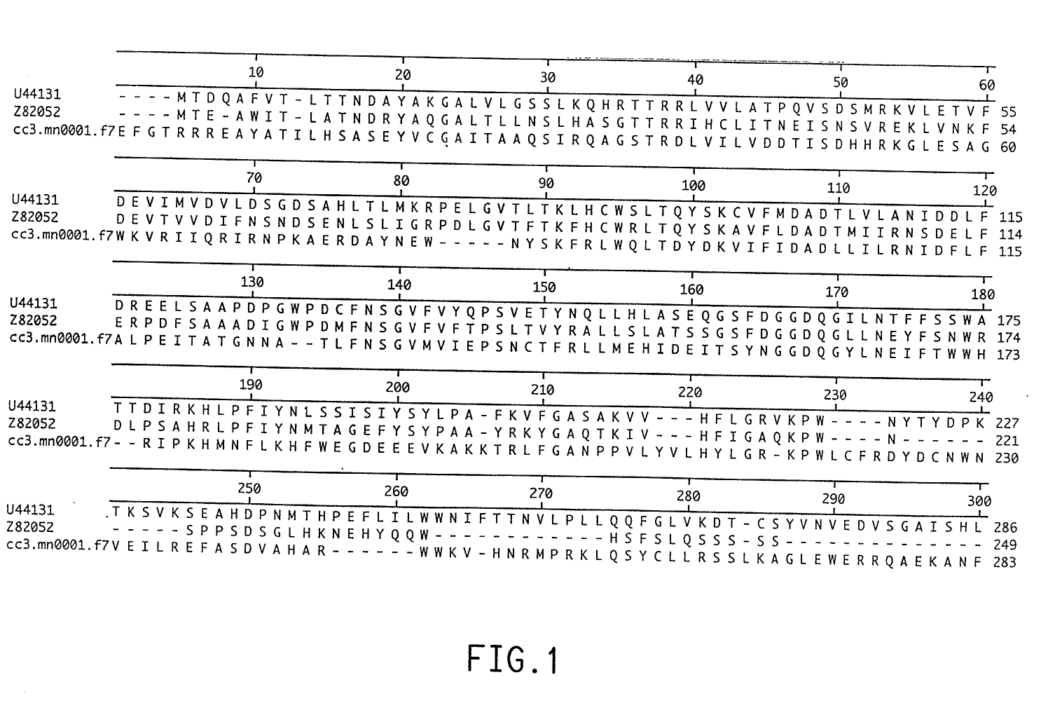

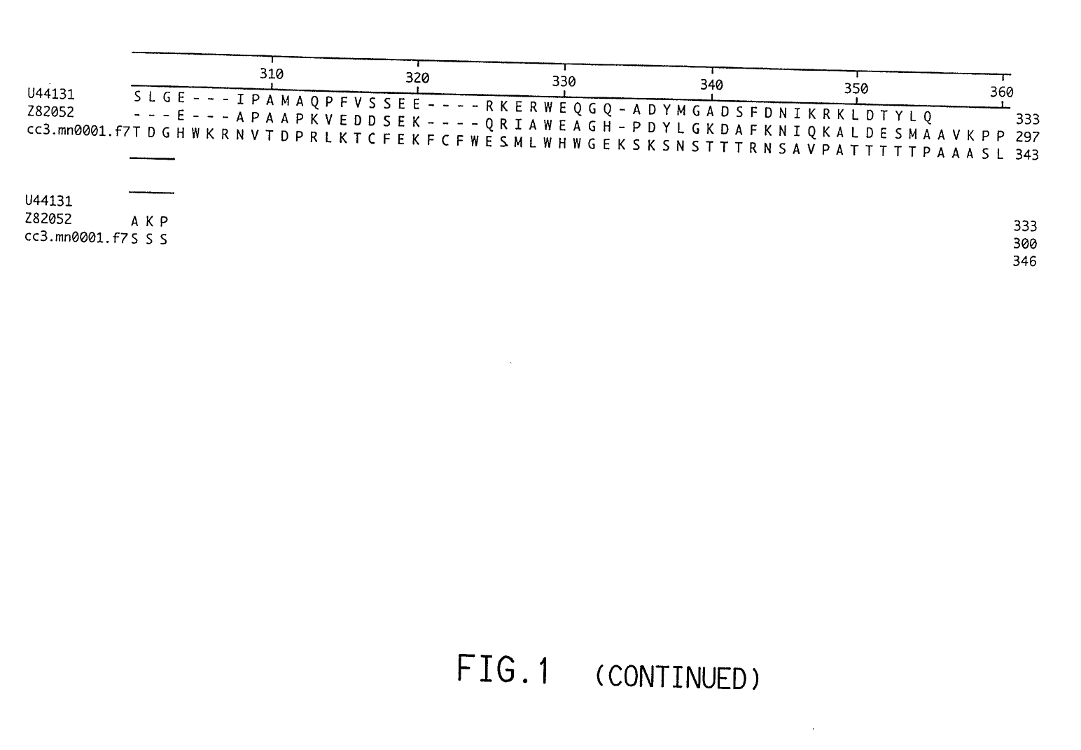

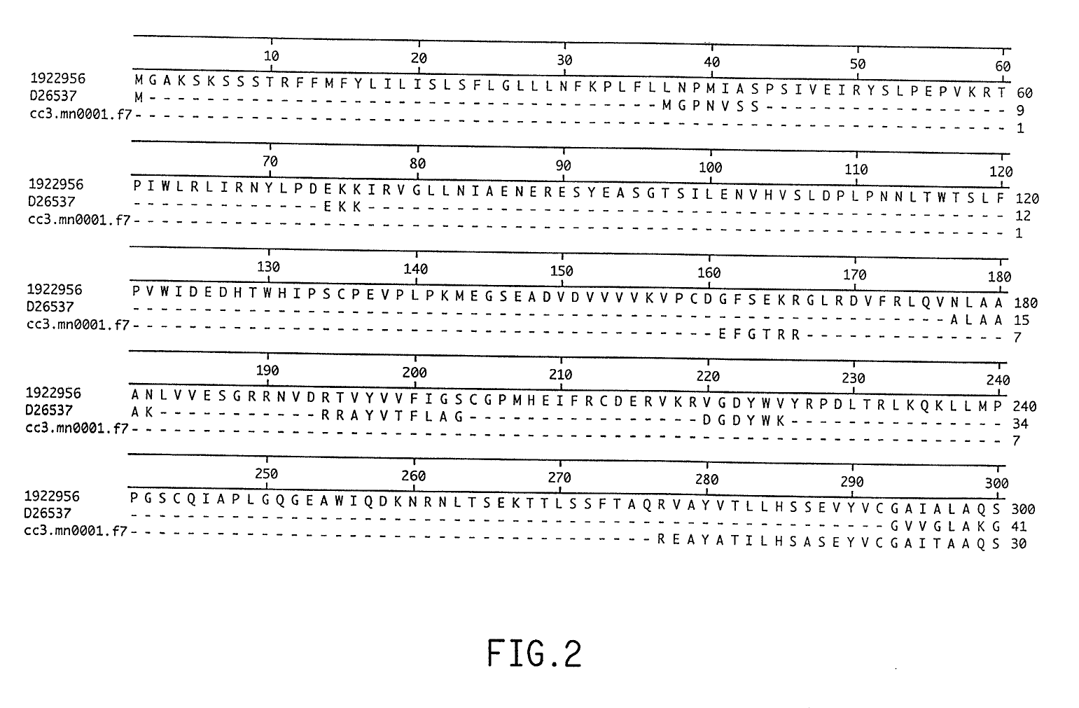

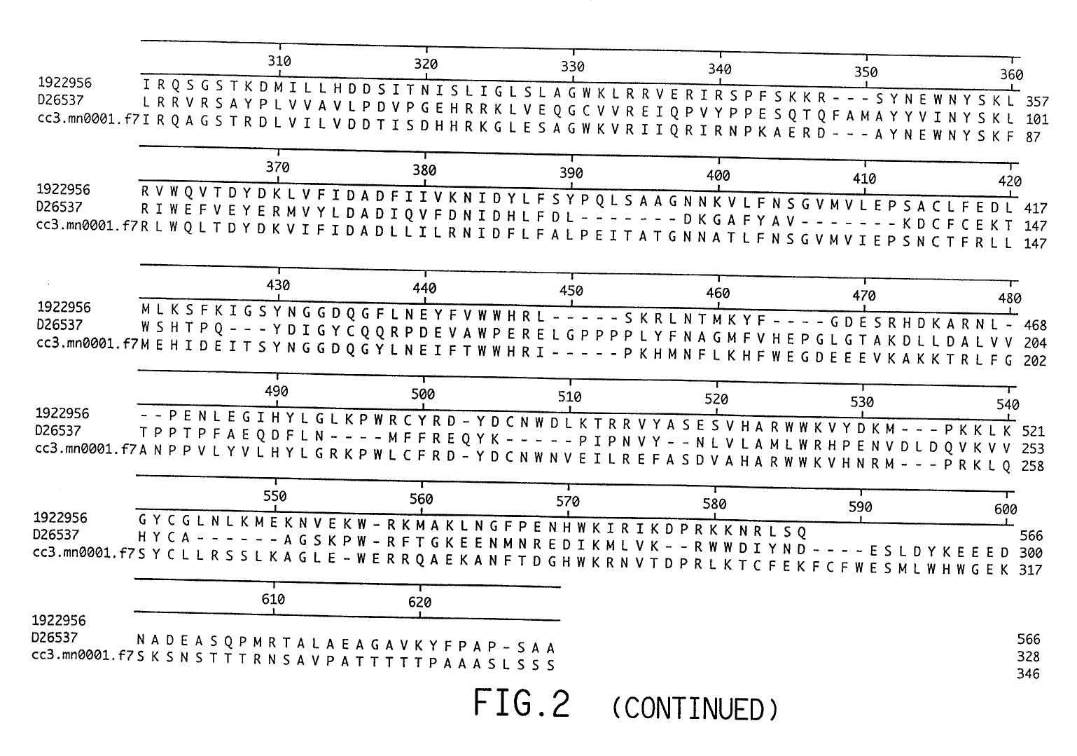

SEQ ID NO:1 is the nucleotide sequence comprising a portion of the cDNA insert in clone cc3.mn0001.f7 encoding a corn glycogenin.

SEQ ID NO:2 is the deduced amino acid sequence of a corn glycogenin derived from the nucleotide sequence of SEQ ID NO:1.

SEQ ID NO:3 is the amino acid sequence encoding the human glycogenin having GenBank Accession No. U44131.

SEQ ID NO:4 is the amino acid sequence encoding the Caenorhabditis elegans glycogenin homolog having EMBL Accession No. Z82052.

SEQ ID NO:5 is the amino acid sequence encoding a conceptual translation of a portion of a genomic clone from Arabidopsis thaliana having GenBank Accession No.1922956.

SEQ ID NO:6 is the amino acid sequence encoding the rice water stress-induced protein having DDJB Accession No. D26537.

SEQ ID NOS:7 is a PCR primer used in the construction of a plasmid vector suitable for expression of the instant corn glycogenin in E. coli.

SEQ ID NOS:8 is a PCR primers used in the construction of a plasmid vector suitable for expression of the instant corn glycogenin in E. coli.

SEQ ID NO:9 is the nucleotide sequence comprising a portion of the cDNA insert in clone cr1n.pk0033.g10 encoding a corn glycogenin.

SEQ ID NO:10 is the deduced amino acid sequence of a corn glycogenin derived from the nucleotide sequence of SEQ ID NO:9.

SEQ ID NO:11 is the nucleotide sequence of a portion of the cDNA insert in clone cta1n.pk0013.e6 encoding a corn glycogenin.

SEQ ID NO:12 is the deduced amino acid sequence of a corn glycogenin derived from the nucleotide sequence of SEQ ID NO:11.

SEQ ID NO:13 is the nucleotide sequence comprising a portion of the cDNA insert in clone rl0n.pk0027.f11 encoding a rice water stress protein.

SEQ ID NO:14 is the deduced amino acid sequence of a water stress protein derived from the nucleotide sequence of SEQ ID NO:13.

SEQ ID NO:15 is the nucleotide sequence comprising a portion of the cDNA insert in clone rr1.pk0070.e9 encoding a rice glycogenin.

SEQ ID NO:16 is the deduced amino acid sequence of a rice glycogenin derived from the nucleotide sequence of SEQ ID NO:15.

SEQ ID NO:17 is the nucleotide sequence a contig assembled from the cDNA inserts in clones wre1n.pk0137.d9 and wre1n.pk0107.h10 encoding a wheat glycogenin.

SEQ ID NO:18 is the deduced amino acid sequence of a glycogenin derived from the nucleotide sequence of SEQ ID NO:17.

SEQ ID NO:19 is the nucleotide sequence comprising a portion of the cDNA insert in clone wlm1.pk0014.g10 encoding a wheat glycogenin.

SEQ ID NO:20 is the deduced amino acid sequence of a glycogenin derived from the nucleotide sequence of SEQ ID NO:19.

SEQ ID NO:21 is the nucleotide sequence comprising a portion of the cDNA insert in clone wl1n.pk0035.h9 encoding a wheat glycogenin.

SEQ ID NO:22 is the deduced amino acid sequence of a glycogenin derived from the nucleotide sequence of SEQ ID NO:21.

SEQ ID NO:23 is the nucleotide sequence comprising a portion of the cDNA insert in clone wl1n.pk0148.f10 encoding a wheat glycogenin.

SEQ ID NO:24 is the deduced amino acid sequence of a wheat glycogenin derived from the nucleotide sequence of SEQ ID NO:23.

SEQ ID NO:25 is the nucleotide sequence of a portion of the cDNA insert in clone wle1n.pk0056.b2 encoding a wheat water stress.

SEQ ID NO:26 is the deduced amino acid sequence of a water stress protein derived from the nucleotide sequence of SEQ ID NO:25.

The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference.

### DETAILED DESCRIPTION OF THE INVENTION

In the context of this disclosure, a number of terms shall be utilized. As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. As used herein, “contig” refers to an assemblage of overlapping nucleic acid sequences to form one contiguous nucleotide sequence. For example, several DNA sequences can be compared and aligned to identify common or overlapping regions. The individual sequences can then be assembled into a single contiguous nucleotide sequence.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotide bases that do not substantially affect the functional properties of the resulting transcript vis-a-vis the ability to mediate alteration of gene expression by antisense or co-suppression technology or alteration of the functional properties of the resulting protein molecule. It is therefore understood that the invention encompasses more than the specific exemplary sequences.

For example, it is well known in the art that antisense suppression and co-suppression of gene expression may be accomplished using nucleic acid fragments representing less that the entire coding region of a gene, and by nucleic acid fragments that do not share 100% identity with the gene to be suppressed. Moreover, alterations in a gene which result in the production of a chemically equivalent amino acid at a given site, but do not effect the functional properties of the encoded protein, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products. Moreover, the skilled artisan recognizes that substantially similar sequences encompassed by this invention are also defined by their ability to hybridize, under stringent conditions (0.1×SSC, 0.1% SDS, 65° C.), with the sequences exemplified herein. Preferred substantially similar nucleic acid fragments of the instant invention are those nucleic acid fragments whose DNA sequences are 80% identical to the DNA sequence of the nucleic acid fragments reported herein. More preferred nucleic acid fragments are 90% identical to the identical to the DNA sequence of the nucleic acid fragments reported herein. Most preferred are nucleic acid fragments that are 95% identical to the DNA sequence of the nucleic acid fragments reported herein.

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to afford specific identification and/or isolation of a nucleic acid fragment comprising the sequence. The instant specification teaches partial or complete amino acid and nucleotide sequences encoding one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequence encoding the corn, rice and wheat glycogenin and water stress proteins as set forth in SEQ ID NOs:2, 10, 12, 14, 16, 18, 20, 22, 24 and 26. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of different lengths may have identical promoter activity.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020).

“Altered levels” refers to the production of gene product(s) in transgenic organisms in amounts or proportions that differ from that of normal or non-transformed organisms.

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propetides may be but are not limited to intracellular localization signals.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels, J. J., (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel, N. (1992) Plant Phys. 100:1627-1632).

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Maniatis”).

This invention relates to corn, rice and wheat cDNAs with homology to glycogenin from mammals and other organisms and rice and wheat cDNAs with homology to water stress proteins from rice. Glycogenin and water stress protein genes from other plants can now be identified by comparison of random cDNA sequences to the corn, rice and wheat glycogenin and water stress protein sequences provided herein.

The nucleic acid fragments of the instant invention may be used to isolate cDNAs and genes encoding homologous glycogenins and water stress proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, other glycogenin or water stress genes, either as cDNAs or genomic DNAs, could be isolated directly by using all or a portion of the instant glycogenin or water stress genes as a DNA hybridization probes to screen libraries from any desired plant employing methodology well known to those skilled in the art. Specific oligonucleotide probes based upon the instant glycogenin or water stress sequences can be designed and synthesized by methods known in the art (Maniatis). Moreover, the entire sequence can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequence. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous glycogenin or water stress protein genes from DNA or RNA. The polymerase chain reaction may also be performed on a library of cloned nucleic acid fragments wherein the sequence of one primer is derived from the instant nucleic acid fragment, and the sequence of the other primer takes advantage of the presence of the polyadenylic acid tracts to the 3′ end of the mRNA precursor encoding plant glycogenin. Alternatively, the second primer sequence may be based upon sequences derived from the cloning vector. For example, the skilled artisan can follow the RACE protocol (Frohman et al., (1988) PNAS USA 85:8998) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., (1989) PNAS USA 86:5673; Loh et al., (1989) Science 243:217). Products generated by the 3′ and 5′ RACE procedures can be combined to generate full-length cDNAs (Frohman, M. A. and Martin, G. R., (1989) Techniques 1:165).

Availability of the instant nucleotide and deduced amino acid sequences facilitates immunological screening cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, R. A. (1984) Adv. Immunol. 36:1; Maniatis).

The nucleic acid fragments of the instant invention may be used to create transgenic plants in which an instant glycogenin or water stress protein is present at higher or lower levels than normal or in cell types or developmental stages in which it is not normally found. This may have the effect of altering starch structure in those cells.

Overexpression of a corn, rice and wheat glycogenin and water stress protein may be accomplished by first constructing a chimeric gene in which a corn, rice and wheat glycogenin or water stress protein coding region is operably linked to a promoter capable of directing expression of a gene in the desired tissues at the desired stage of development. For reasons of convenience, the chimeric gene may comprise a promoter sequence and translation leader sequence derived from the same gene. 3′ Non-coding sequences encoding transcription termination signals must also be provided. The instant chimeric genes may also comprise one or more introns in order to facilitate gene expression.

A plasmid vector comprising the instant chimeric gene is then constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications it may be useful to direct the glycogenin or water stress protein protein to different cellular compartments, or to facilitate its secretion from the cell. It is thus envisioned that the chimeric gene described above may be further supplemented by altering the coding sequence to encode a glycogenin or water stress protein with appropriate intracellular targeting sequences such as transit sequences (Keegstra, K. (1989) Cell 56:247-253), signal sequences or sequences encoding endoplasmic reticulum localization (Chrispeels, J. J., (1991) Ann. Rev. Plant Phys. Plant Mol. Biol. 42:21-53), or nuclear localization signals (Raikhel, N. (1992) Plant Phys.100:1627-1632) added and/or with targeting sequences that are already present removed. While the references cited give examples of each of these, the list is not exhaustive and more targeting signals of utility may be discovered in the future. It may also be desirable to reduce or eliminate expression of the glycogenin or water stress protein gene in plants for some applications. In order to accomplish this, a chimeric gene designed for co-suppression of glycogenin can be constructed by linking the glycogenin gene or gene fragment to a plant promoter sequences. Alternatively, a chimeric gene designed to express antisense RNA for all or part of the glycogenin gene can be constructed by linking the glycogenin gene or gene fragment in reverse orientation to a plant promoter sequences. Either the co-suppression or antisense chimeric gene could be introduced into plants via transformation wherein expression of the endogenous glycogenin gene is reduced or eliminated.

Corn, rice and wheat glycogenin or water stress proteins produced in heterologous host cells, particularly in the cells of microbial hosts, can be used to prepare antibodies to the protein by methods well known to those skilled in the art. The antibodies are useful for detecting corn, rice and wheat glycogenin or water stress proteins in situ in cells or in vitro in cell extracts. Preferred heterologous host cells for production of a corn, rice or wheat glycogenin and water stress protein are microbial hosts. Microbial expression systems and expression vectors containing regulatory sequences that direct high level expression of foreign proteins are well known to those skilled in the art. Any of these could be used to construct chimeric genes for production of a corn, rice and wheat glycogenin or water stress proteins. These chimeric genes could then be introduced into appropriate microorganisms via transformation to provide high level expression of a corn, rice and wheat glycogenin and water stress proteins. An example of a vector for high level expression of a corn, rice and wheat glycogenin or water stress protein in a bacterial host is provided (Example 4).

All or a portion of the nucleic acid fragments of the instant invention may also be used as probes for genetically and physically mapping the genes that they are a part of, and as markers for traits linked to expression of a corn, rice and wheat glycogenin or water stress protein. Such information may be useful in plant breeding in order to develop lines with desired starch phenotypes.

For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et at., (1987) Genomics 1:174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein, D. et al., (1980) Am. J. Hum. Genet. 32:314-331).

The production and use of plant gene-derived probes for use in genetic mapping is described in R. Bematzky, R. and Tanksley, S. D. (1986) Plant Mol. Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel, J. D., et al., In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping (Trask, B. J. (1991) Trends Genet. 7:149-154). Although current methods of FISH mapping favor use of large clones (several to several hundred KB; see Laan, M. et al. (1995) Genome Research 5:13-20), improvements in sensitivity may allow performance of FISH mapping using shorter probes.

A variety of nucleic acid amplification-based methods of genetic and physical mapping may be carried out using the instant nucleic acid sequences. Examples include allele-specific amplification (Kazazian, H. H. (1989) J. Lab. Clin. Med. 114(2):95-96), polymorphism of PCR-amplified fragments (CAPS; Sheffield, V. C. et al. (1993) Genomics 16:325-332), allele-specific ligation (Landegren, U. et al. (1988) Science 241:1077-1080), nucleotide extension reactions (Sokolov, B. P. (1990) Nucleic Acid Res. 18:3671), Radiation Hybrid Mapping (Walter, M. A. et al. (1997) Nature Genetics 7:22-28) and Happy Mapping (Dear, P. H. and Cook, P. R. (1989) Nucleic Acid Res. 1 7:6795-6807). For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

Loss of function mutant phenotypes may be identified for the instant cDNA clones either by targeted gene disruption protocols or by identifying specific mutants for these genes contained in a maize population carrying mutations in all possible genes (Ballinger and Benzer, (1989) Proc. Natl. Acad. Sci USA 86:9402; Koes et al., (1995) Proc. Natl. Acad. Sci USA 92:8149; Bensen et al., (1995) Plant Cell 7:75). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the plant gene encoding the corn, rice and wheat glycogenin or water stress protein. Alternatively, the instant nucleic acid fragment may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous gene encoding a corn, rice and wheat glycogenin or water stress protein can be identified and obtained. This mutant plant can then be used to determine or confirm the natural function of the corn, rice and wheat glycogenin or water stress protein gene product.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### EXAMPLE 1

### Composition of Corn, Rice and Wheat cDNA Librarys: Isolation and Sequencing of cDNA Clones

A cDNA library representing mRNAs from corn embryogenic callus derived from corn embryos obtained from Zea mays LH132 corn plants (library desigantion: cc3) was prepared. The cDNA library was prepared in a Uni-ZAP™ XR vector according to the manufacturer's protocol (Stratagene Cloning Systems, La Jolla, Calif.). Conversion of the Uni-ZAP™ XR library into a plasmid library was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted corn cDNA sequences. Amplified insert DNAs were sequenced in dye-primer sequencing reactions to generate partial cDNA sequences (expressed sequence tags or “ESTs”; see Adams, M. D. et al., (1991) Science 252:1651). The resulting ESTs were analyzed using a Perkin Elmer Model 377 fluorescent sequencer. cDNA libraries representing mRNAs from various other corn, rice and wheat tissues were also prepared as describe above. The characteristics of these libraries are described below.

TABLE 1
cDNA Libraries from Corn, Rice and Wheat

| Library | Tissue | Clone |
|---|---|---|
| cr1n | Corn Root From 7 Day Seedlings | cr1n.pk0033.g10 |
| | Grown In Light* | |
| cta1n | Corn Tassel* | cta1n.pk0027.e11 |
| r10n | Rice 15 Day Leaf* | r10n.pk0027.f11 |
| rr1 | Rice Root Two Week Old Developing | rr1.pk0070.e9 |
| | Seedling | |
| wre1n | Wheat Root From 7 Day Old | wre1n.pk0137.d9 |
| | Etiolated Seedling* | |
| | | wre1n.pk0107.h10 |
| w11n | What Leaf Obtained From 7 Day Old | w11n.pk0035.h9 |
| | Etiolated Seedling* | |
| | | ww11n.pk0148.f10 |
| w131n | Wheat Leaf From 7 Day Old | w1e1n.pk0056.b2 |
| | Etiolated Seedling* | |
| w1m1 | Wheat Seedling 1 Hour After Inocula- | w1m1.pk0014.g10 |
| | tion With Erysiphe graminis | |

*These libraries were normalized essentially as described in U.S. Pat. No. 5,482,845

### EXAMPLE 2

### Identification and Characterization of cDNA Clones

ESTs encoding glycogenin were identified by conducting a BLAST (Basic Local Alignment Search Tool; Altschul, S. F., et al., (1990) J. Mol. Biol. 215:403-410; see also www.ncbi.nlm.nih.gov/BLAST/) search for similarity to sequences contained in the GenBank database. The cDNA sequences obtained in Example 1 was analyzed for similarity to all publicly available DNA sequences contained in the GenBank Database using the BLASTN algorithm provided by the National Center for Biotechnology Information (NCBI). The DNA sequences were translated in all reading frames and compared for similarity to all publicly available protein sequences contained in the GeneBank Database using the BLASTX algorithm (Gish, W. and States, D. J. (1993) Nature Genetics 3:266-272) provided by the NCBI.

The BLASTX search using clone cc3.mn0001.f7 revealed similarity of the protein encoded by the cDNA to human glycogenin (GenBank Accession No. U31525; logP=23.47). The sequence of the entire cDNA insert in clone cc3.mn0001.f7 was then determined and is depicted in SEQ ID NO:1. The corresponding amino acid sequence of the corn glycogenin protein is shown in SEQ ID NO:2. The amino acid sequence was then analyzed for similarity to all publically available sequences using the BLASTP algorithm provided by the NCBI. The BLASTP search using the sequence depicted in SEQ ID NO:2 revealed significant homology to human glycogenin (GenBank Accession No. U44131; logP=19.62) and a Caenorhabditis elegans glycogenin homolog (EMBL Accession No. Z82052; logP=21.60). The BLASTP search also revealed homology of the instant corn EST to two plant peptide sequences: a conceptual translation of a portion of a genomic clone from Arabidopsis thaliana (GenBank Accession No. 1922956; logP=116.77) with homology to glycogenin, and a rice (Oryza sativa) protein induced by water stress (DDJB Accession No. D26537; logP=16.89). The amino acid sequence of the instant corn glycogenin shows approximately 19.2, 20.3, 43.1 and 16.8% sequence similarity (calculated using Clustal Method and the PAM250 Weight Table (DNASTAR Inc., Madison, Wis.)) to the human, C. elegans, Arabidopsis and rice sequences, respectively. Sequence alignments and BLAST scores and probabilities indicate that the instant nucleic acid fragment encodes a corn glycogenin enzyme.

### EXAMPLE 3

### Characterization of cDNA Clones Encoding Other Glycogenins or Water Stress Proteins

The BLASTX search using the EST sequences from several clones revealed similarity of the proteins encoded by the cDNAs to glycogenins or water stress proteins from different organisms. The BLAST results for each of these ESTs are shown in Table 2:

TABLE 2
BLAST Results for Clones Encoding Polypeptides Homologous
to Glycogenin or Water Stress Proteins

| | | | GenBank | |
|---|---|---|---|---|
| | | | | Blast |
| | | | Accession | pLog |
| Clone | Protein | Organism | No. | score |
| cr1n.pk0033.g10 | Glycogenin | Rhodobacter | M89780 | 10.57 |
| | | sphaeroides | | |
| r10n.pk0027.f11 | Water Stress | Oryza sativa | D26537 | 39.36 |
| | Protein | | | |
| rr1.pk0070.e9 | Water Stress | Caenorhabditis | U64599 | 17.59 |
| | Protein | elegans | | |
| w11n.pk0035.h9 | Glycogenin | Caenorhabditis | U64599 | 6.24 |
| | | elegans | | |
| w11n.pk0148.f10 | Glycogenin | Caenorhabditis | U64599 | 13.85 |
| | | elegans | | |
| w1e1n.pk0056.b2 | Glycogenin | Caenorhabditis | U64599 | 6.72 |
| | | elegans | | |
| w1m1.pk0014.g10 | Water Stress | Oryza sativa | D26537 | 22.51 |
| | Protein | | | |

BLAST scores and probabilities indicate that the instant nucleic acid fragments encode portions of glycogenin or water stress proteins. These sequences represent additional, heretofore unrecognized corn sequences encoding glycogenin. In addition, the wheat clones described above represent the first wheat sequences encoding a glycogenin or water stress protein. Clones rl0n.pk0027.f11 and rr1.pk0070.e9 appear to encode proteins that belong to the water stress protein gene family but have not been previously identified in rice. This conclusion is based on the fact that rl0n.pk0027.f1·1 and rr1.pk0070.e9 bear little or no homology to known rice water stress proteins genes as evidenced by their low pLog scores.

Two other clones, cta1n.pk0013.e6 and wre1n.pk0137.d9, were identified as encoding glycogenin by their homology to cc3.mm0001.f7. When compared to cc3.mm0001.f 7by BLAST, they had pLog values of 50.69 for cta1n.pk0013.e6 and 41.30 for wre1n.pk0107.h10. An additional wheat clone, wre1n.pk0107.h10, was identified by BLAST homology to wre1n.pk0137.d9. When compared, wre1n.pk0107.h10 and wre1n.pk0137.d9 were found have an overlapping region of nearly 100% identity. Using this homology it was possible to align these clones and assemble a contig (a contig is an assemblage of overlapping nucleic acid sequences to form one contiguous nucleotide sequence). The individual sequences were assembled into a unique contiguous nucleotide sequence encoding a unique wheat glycogenin protein. The SEQ ID NOs for each the above clones and the wheat glycogenin contig are shown in Table 3:

TABLE 3
Sequence Identification Numbers for Clones Encoding
Polypeptides Homologous
to Glycogenin or Water Stress Proteins

| | SEQ ID NOs. | |
|---|---|---|
| Clone | Nucleotide Sequence | Amino Acid Sequence |
| cr1n.pk0033.g10 | 9 | 10 |
| cta1n.pk0013.e6 | 11 | 12 |
| r10n.pl0027.f11 | 13 | 14 |
| rr1.pk0070.e9 | 15 | 16 |
| Contig composed of: | | |
| wre1n.pk0137.d9 | | |
| wre1n.pk0107.h10 | | |
| w1m1.pk0014.g10 | 19 | 20 |
| w11n.pk0035.h9 | 21 | 22 |
| w11n.pk0148.f10 | 23 | 24 |
| w1e1n.pk0056.b2 | 25 | 26 |

### EXAMPLE 4

### Expression of Chimeric Genes in Plant Cells

A chimeric gene comprising a corn, rice or wheat glycogenin or water stress protein cDNA in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone comprising a corn, rice or wheat glycogenin or water stress protein using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a 100 uL volume in a standard PCR mix consisting of 0.4 mM of each oligonucleotide and 0.3 pM of target DNA in 10 mM Tris-HCl, pH 8.3, 50 mM KCl, 1.5 mM MgCl₂, 0.001% w/v gelatin, 200 mM dGTP, 200 mM dATP, 200 mM dTTP, 200 mM dCTP and 0.025 unit Amplitaq™ DNA polymerase. Reactions are carried out in a Perkin-Elmer Cetus Thermocycler™ for 30 cycles comprising 1 minute at 95° C., 2 minutes at 55° C. and 3 minutes at 72° C., with a final 7 minute extension at 72° C. after the last cycle. The amplified DNA is then digested with restriction enzymes NcoI and SmaI and fractionated on a 0.7% low melting point agarose gel in 40 mM Tris-acetate, pH 8.5, 1 mM EDTA. The appropriate band can be excised from the gel, melted at 68° C. and combined with a 4.9 kb NcoI-SmaI fragment of the plasmid pML103. Plasmid pML103 has been deposited under the terms of the Budapest Treaty at ATCC (American Type Culture Collection, 12301 Parklawn Drive, Rockville, Md. 20852), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL 1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U. S. Biochemical). The resulting plasmid construct would comprise a chimeric gene encoding, in the 5′ to 3′ direction, the maize 27 kD zein promoter, the corn, rice or wheat glycogenin or water stress protein cDNA fragment, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al., (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al., (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., (1990) Bio/Technology 8:833-839).

Starch extracted from single seeds obtained from plants transformed with the chimeric gene can then be analyzed. Seeds can be steeped in a solution containing 1.0% lactic acid and 0.3% sodium metabisulfite, pH 3.8, held at 52° C. for 22-24 h. Seeds are then drained, rinsed and homogenized individually in 8-9 mL of a solution of 100 mM NaCl. Five mL of toluene are added to each tube and vigorously shaken twice for 6 minutes using a paint mixer, and allowed to settle for 30 minutes. Two mL of 100 mM NaCl is sprayed onto the solution, allowed to settle for 30 minutes, and the protein-toluene layer is aspirated off. The toluene wash step is repeated. Twelve mL water is added and shaken in a paint shaker for 45 seconds. This solution is centrifuged for 10 minutes and the water is removed. The water wash is repeated, followed by a final wash with 12 mL of acetone. After shaking and centrifugation steps, the acetone is drained and allowed to evaporate for 1 h. Starch extracts are incubated in a 40° C. oven overnight.

Extracted starches can be enzymatically debranched as follows. Seven mg of each starch sample is added to a screw cap test tube containing 1.1 mL of water. The tubes are heated to 120° C. for 30 minutes and then placed in a water bath at 45° C. Debranching solution can be prepared by diluting 50 μL of isoamylase (5×10⁶ units/mL; Sigma) per mL of 50 mM NaOAc buffer, pH 4.5. Forty μL of debranching solution is added to each starch sample, and the samples are incubated in a water bath at 45° C. for 3 h. The debranching reaction is stopped by heating samples to 110° C. for 5 minutes. Debranched starch samples can then be lyophilized and redisolved in DMSO.

One hundred μL of each debranched starch can then be analyzed by gel permeation chromotography (GPC). One hundred μL of each debranched starch is injected and chromatographed by passage through two GPC columns (Mixed Bed-C; Polymer Labs) arranged in series. Chromatography is performed at 100° C. and samples are eluted with DMSO at a flow rate of 1.0 mL/min. Chromatographic samples are collected at 25 minute intervals. A refractive index detector (Waters) can be used for detection, and data can be collected and stored with the aid of a computer running Chemstation Software (version A.02.05; Hewlett-Packard).

Retention times of collected samples may then be compared to retention times of pullulan standards (380K, 100K, 23.7K, 5.8K, 728 and 180 mw). The proportion of the total starch is determined for twenty-four ranges of degree of polymerization (DP) spanning both the amylose and amylopectin portions of the chromatogram. The percentage area in appropriate DP ranges is used to determine values for A & B1, B2, B3 and B4+ chains of the amylopectin portion of the chromatogram. The proportion of the total area above DP 150 is used to determine amylose content.

Amylopectin is typically described by its distribution of branch chains in the molecule. The amylopectin molecule is comprised of alternating crystalline and amorphous regions. The crystalline region is where many of the branch points (α-1,6 linkages) occur, while the amorphous region is an area of little to no branching and few branch chains. The type of chain may be designated as A or B. A chains are unbranched and span a single crystalline region. B1 chains also span a single crystalline region but are branched. B2, B3 and B4+ chains are branched and span 2, 3 and 4 or more crystalline regions, respectively (Hizukuri (1986) Carbohydrate Res. 147:342-347). The relative area under the amylopectin portion of the chromatograms can be used to determine the area percentage of the A & B1, B2, B3 and B4+ chains.

Starches derived from plants transformed with the chimeric gene can also be tested for functionality by techniques well known to those skilled in the art. For example, starch can be extracted from dry mature kernels from transformed plants. Fifteen g of kernels are weighed into a 50 mL Erlenmeyer flask and steeped in 50 mL of steep solution (same as above) for 18 h at 52° C. The kernels are drained and rinsed with water. The kernels are then homogenized using a 20 mm Polytron probe (Kinematica GmbH; Kriens-Luzern, Switzerland) in 50 mL of cold 50 mM NaCl. The homogenate is filtered through a 72 micron mesh screen. The filtrate is brought up to a total volume of 400 mL with 50 mM NaCl and an equal volume of toluene is added. The mixture is stirred with a magnetic stir bar for 1 h at sufficient speed to completely emulsify the two phases. The emulsion is allowed to separate overnight in a covered beaker. The upper toluene layer is aspirated from the beaker and discarded. The starch slurry remaining in the bottom of the beaker is resuspended, poured into a 250 mL centrifuge bottle and centrifuged 15 minutes at 25,000 RCF. The supernatant is discarded and the starch is washed sequentially with water and acetone by shaking and centrifuging as above. After the acetone wash and centrifugation the acetone is decanted and the starch allowed to dry overnight in a fume hood at room temperature.

A Rapid Visco Analyzer (Newport Scientific; Sydney, Australia) with high sensitivity option and Thermocline software can then be used for pasting curve analysis. For each line, 1.50 g of starch is weighed into the sample cup and 25 mL of phosphate/citrate buffer (pH 6.50) containing 1% NaCl was added. Pasting curve analysis can be performed using the following temperature profile: idle temperature 50° C., hold at 50° C. for 0.5 minutes, linear heating to 95° C. for 2.5 minutes, linear cooling to 50° C. over 4 minutes, hold at 50° C. for four minutes.

Results of the Rapid Visco Analyzer pasting analysis may demonstrate that the starch produced by lines transformed with the chimeric gene differ in its pasting properties both from normal dent starch. This result may demonstrate that the alteration of starch fine structure produced by altering expression of a corn, rice or wheat glycogenin or water stress protein can create a starch of novel functionality.

The size of the individual starch granules is an important component of milling yield, as well as a contributing factor in starch functionality. Because decreases of increases in the amount of glycogenin primer may reduce or increase, respectively, the number of starch granules initiated, the resulting granules may be expected to be altered in size relative to normal maize starch granules. Starch extracted from individual kernels can be subjected to Particle Size Analysis (PSA). 7.5 mg of starch is dispersed in dispersing solution comprising 0.2% Triton X-100 in water (v/v) and sonicated for 15 minutes. The particle size of the dispersion is then measured using a PSA2010 Particle Size Analyzer (Galai Production Ltd.) equipped with a BCM-1 Cell Module. Particle size measurements are made according to the manufacturer's instructions. Changes in granule size may indicate altered starch functionality or millability.

### EXAMPLE 5

### Expression of Corn Glycogenin in E. coli

For expression in E. coli, the EST clone cc3.mn0001.f7 was placed into the pET24d T7 expression vector (Novagen) by PCR amplification using primers depicted in SEQ ID NO:7 and SEQ ID NO:8. For PCR, Vent™ DNA polymerase (New England Biolabs) was used with an additional 2 μL of 100 mM magnesium sulfate added to each 100 μL reaction. The 5′ primer has the sequence shown in SEQ ID NO:7 and consists of bases 26 to 46 of SEQ ID NO:1, additional bases 5′-catgccatgg-3′ added to encode an Nco I site in the primer and four additional 5′ bases to enhance the restriction enzyme recognintion of the encoded Nco I site. The 3′ primer has the sequence shown in SEQ ID NO:8 and consists of the reverse complement of bases 625 to 646 in pBluescript-SK (Stratagene). The PCR reaction comprised for 25 cycles using the following protocol: 55° C. annealing temperature and 1.5 minute extension time. A product of about 1400 base pairs was obtained and purified using Wizard™ PCR purification kit (Perkin-Elmer). Four micrograms of the PCR product was digested for 18 hours at 37° C. with NcoI and XhoI. The digested DNA was deproteinated by extraction with an equal volume of 1:1 phenol:chloroform, extraction of the upper layer of the phenol:chloroform separation with 1 volume of chloroform, and precipitation with ethanol. One microgram of digested PCR product was then ligated with 200 ng of pET24d T7 expression vector (Novogen) that had also been previously digested with NcoI and XhoI. The ligation mixture was used to transform electrocompetent BL21 (DE3) (Novagen) E. coli cells and transformants were selected by growth on plates containing 50 mg/L kanamycin. Eighteen single colonies from the transformation plate were chosen to inoculate 3 mL cultures of 2×YT media containing 50 mg/L kanamycin in preparation for plasmid purification. Insertion of the PCR product in the expression vector was determined by restriction enzyme analysis using NcoI and XhoI.

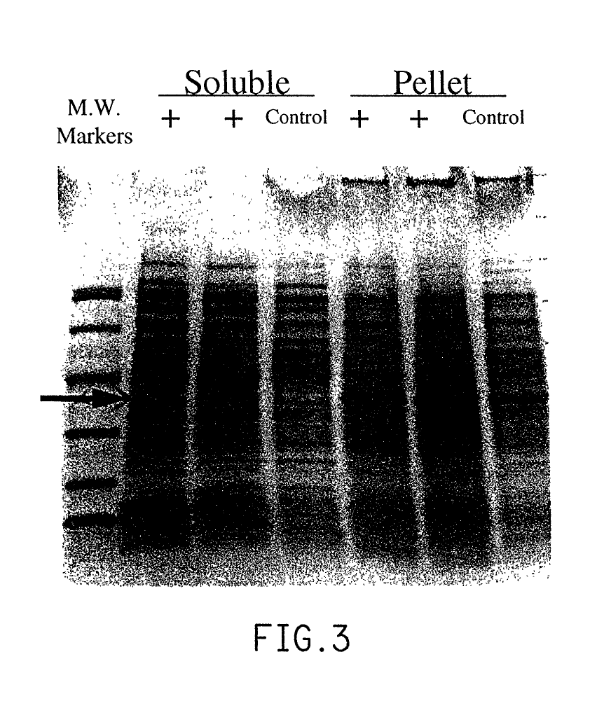

Three kanamycin resistant clones were chosen for inoculation of overnight cultures. Two of the clones contained the PCR generated EST cc3.mn0001.f7 insert, while the third clone was an empty pET24d vector to act as a control. The overnight cultures which were grown at 30° C. in 2×YT media containing 50 mg/L kanamycin were diluted two fold with fresh media, allowed to re-grow for 1 h, then induced by adding isopropyl-thiogalactoside to 1 mM final concentration. Following a 3 h induction period, cells were harvested by centrifugation and re-suspended in 50 μL of 50 mM Tris-HCl at pH 8.0 containing 0.1 mM DTT and 0.2 mM phenyl methylsulfonyl fluoride. A small amount of 1 mm glass beads were added and the mixture was sonicated 3 times for about 5 seconds each time with a microprobe sonicator. The mixture was centrifuged and the protein concentration of the supernatant and pellet were determined. One μg of protein from the soluble fraction and pellet of each clonal culture was separated by SDS-polyacrylamide gel electrophoresis. The cultures contiaing the corn glycogenin cDNA insert produced an additional protein band of about 42 kilodaltons in mass predominately in the pellet fraction with a small percentage in the soluble fraction (FIG. 3).

What is claimed is:

1. An isolated nucleic acid fragment comprising a member selected from the group consisting of:
(a) an isolated nucleic acid fragment encoding all or a substantial portion of the amino acid sequence set forth in a member selected from the group consisting of SEQ ID NO:2, 10, 12, 14, 16, 18, 20, 22, 24 and 26;
(b) an isolated nucleic acid fragment that is substantially similar to an isolated nucleic acid fragment encoding all or a substantial portion of the amino acid sequence set forth in a member selected from the group consisting of SEQ ID NO:2, 10, 12, 14, 16, 18, 20, 22, 24 and 26 ; and
(c) an isolated nucleic acid fragment that is complementary to (a) or (b).

2. The isolated nucleic acid fragment of claim 1 wherein the nucleotide sequence of the fragment is set forth in a member selected from the group consisting of SEQ ID NO:1, 9, 11, 13, 15, 17, 19, 21, 23 and 25.

3. A chimeric gene comprising the nucleic acid fragment of claim 1 operably linked to suitable regulatory sequences.

4. A transformed host cell comprising the chimeric gene of claim 3.

5. A method of altering the level of expression of a plant glycogenin or water stress protein in a host cell comprising:
(a) transforming a host cell with the chimeric gene of claim 3; and
(b) growing the transformed host cell produced in step (a) under conditions that are suitable for expression of the chimeric gene
wherein expression of the chimeric gene results in production of altered levels of a plant glycogenin or water stress protein in the transformed host cell.

6. A method of obtaining a nucleic acid fragment encoding all or substantially all of the amino acid sequence encoding a plant glycogenin or water stress protein comprising:
(a) probing a cDNA or genomic library with the nucleic acid fragment of claim 1;
(b) identifying a DNA clone that hybridizes with the nucleic acid fragment of claim 1; and
(c) sequencing the cDNA or genomic fragment that comprises the clone identified in step (c)
wherein the sequenced nucleic acid fragment encodes all or substantially all of the amino acid sequence encoding a plant glycogenin or water stress protein.

7. A method of obtaining a nucleic acid fragment encoding a portion of an amino acid sequence encoding a plant glycogenin or water stress protein comprising:
(a) synthesizing an oligonucleotide primer corresponding to a portion of the sequence set forth in a member selected from the group consisting of SEQ ID NO:1, 9, 11, 13, 15, 17, 19, 21, 23 and 25; and
(b) amplifying a cDNA insert present in a cloning vector using the oligonucleotide primer of step (a) and a primer representing sequences of the cloning vector
wherein the amplified nucleic acid fragment encodes a portion of an amino acid sequence encoding a plant glycogenin or water stress protein.

8. The product of the method of claim 6

9. The product of the method of claim 7.
